(19) United States
(12) Patent Application Publication
(10) Pub. No.: US 2023/0383367 A1
(43) Pub. Date: Nov. 30, 2023

(54) METHOD AND KIT FOR SIMULTANEOUSLY DETECTING A PLURALITY OF DIFFERENT PATHOGENS IN A SAMPLE

(71) Applicant: XEBIOS DIAGNOSTICS GROUP GMBH, Duesseldorf (DE)

(72) Inventor: Ludwig Decker, Eelde (NL)

(73) Assignee: XEBIOS DIAGNOSTICS GROUP GMBH, Duesseldorf (DE)

(21) Appl. No.: 18/200,582

(22) Filed: May 23, 2023

(30) Foreign Application Priority Data
May 30, 2022 (DE) 10 2022 113 596.9

(51) Int. Cl. C12Q 1/689; C12Q 1/6806; C12Q 1/686; C12N 1/06

(52) U.S. Cl. CPC C12Q 1/689; C12Q 1/6806; C12Q 1/686; C12N 1/06

(57) ABSTRACT

A method for detecting a plurality of different pathogens in a sample. The method includes a pre-enrichment step of adding the sample to a pre-enrichment medium so as to obtain a pre-enriched sample, a lysis step of breaking open cell walls of the plurality of different pathogens in the pre-enriched sample so as to provide a lysate, and a detection step of detecting the plurality of different pathogens via a multiplex PCR investigation. The pre-enrichment step, the lysis step, and the detection step are performed in the order specified. The lysis step is terminated before the detection step without increasing temperature.

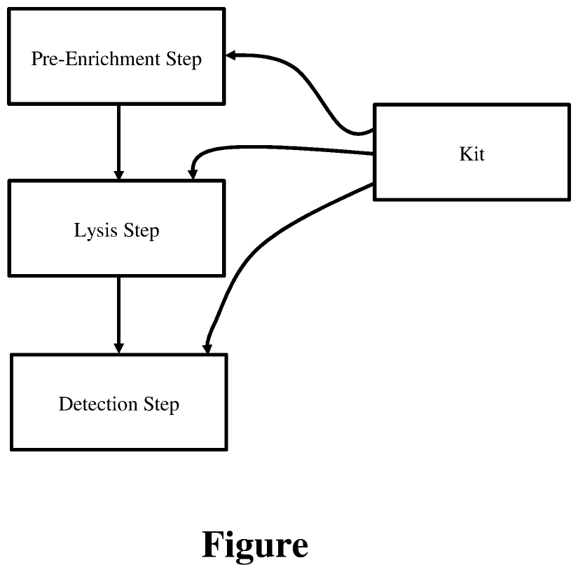

### CROSS REFERENCE TO PRIOR APPLICATIONS

Priority is claimed to German Patent Application No. DE 10 2022 113 596.9, filed May 30, 2022. The entire disclosure of said application is incorporated by reference herein.

### FIELD

The present invention relates to a method for simultaneously detecting a plurality of different pathogens in a sample via a multiplex PCR investigation. The present invention also relates to a kit for simultaneously detecting a plurality of different pathogens in a sample via a multiplex PCR investigation.

### BACKGROUND

Investigations of samples for detecting pathogens have previously been described. For example, WO 2016/164,407 A2 describes a method for detecting a plurality of pathogens in a sample. The pathogens are multiplied in a pre-enrichment medium. Cell walls are subsequently broken open in a first lysis step and in a second lysis step in order to produce a lysate that is suitable for being investigated with the aid of a multiplex PCR method. The second lysis step here takes place at a higher temperature than the first step.

### SUMMARY

An aspect of the present invention is to provide a method which provides an improved sequence compared to the prior art and to provide a kit with which such a method can be performed.

In an embodiment, the present invention provides a method for detecting a plurality of different pathogens in a sample. The method includes a pre-enrichment step comprising adding the sample to a pre-enrichment medium so as to obtain a pre-enriched sample, a lysis step comprising breaking open cell walls of the plurality of different pathogens in the pre-enriched sample so as to provide a lysate, and a detection step comprising detecting the plurality of different pathogens via a multiplex PCR investigation. The pre-enrichment step, the lysis step, and the detection step are performed in an order set forth. The lysis step is terminated before the detection step without increasing a temperature.

The present invention also provides a kit for detecting a plurality of different pathogens using the method. The kit includes a pre-enrichment medium for providing the pre-enriched sample in the pre-enrichment step, a lysis reagent for breaking open the cell walls of the plurality of different pathogens in the lysis step, and a PCR reagent for performing the multiplex PCR investigation in the detection step.

### BRIEF DESCRIPTION OF THE DRAWING

The present invention is described in greater detail below on the basis of embodiments and of the drawings in which:

The FIGURE shows a schematic representation of a method sequence according to an embodiment of the present invention with a kit according to an embodiment of the present invention.

### DETAILED DESCRIPTION

For carrying out the method, a sample can, for example, be removed from a substance to be investigated. It is conceivable that this substance can, for example, be a food such as a meat product or a milk product. A meat product may, for example, be beef, a cut meat, sausage, ham, or any other food in which meat is used. A milk product may, for example, be milk, quark, yogurt, or any other food in which milk is used. It is also conceivable that the substance be animal feed.

The weight of the sample can, for example, be more than 1 g and less than 100 g, for example, more than 10 g and less than 50 g, for example, 25 g, in weight.

In order to detect the plurality of different pathogens, the present invention provides that the sample first passes through a pre-enrichment step for pre-enrichment in a pre-enrichment medium. In a subsequent lysis step, the cell walls of the pathogens in the pre-enriched sample are then broken open. This serves to release the DNA of the pathogens. The breaking of the cell walls of the pathogens is also referred to as “lysis”. Lysis can be brought about in different ways. For example, the cell walls can be broken open with an increased temperature during a thermal lysis. In a chemical lysis, cell disruption occurs, for example, via the addition of chaotropic salts, detergents, or other chemical auxiliary agents. An enzymatic lysis uses an enzyme, for example, proteinase K and/or lysozyme, for cell disruption. Proteinase K also contributes to switching off nucleases that are released during cell disruption. A mechanical lysis is also possible in which the cell disruption is brought about, for example, via mechanical stress on the cell walls with so-called beads. Beads are small spheres of a hard material, for example, on a ceramic, glass, or zirconium basis. It is in particular provided that, in the lysis step, first a thermal lysis and subsequently a chemical lysis be used for cell disruption. An enzymatic lysis and/or a mechanical lysis can additionally contribute to cell disruption in the lysis step.

If the lysis step is completed, the plurality of pathogens are detected in a detection step via a multiplex PCR investigation. For this purpose, the lysate is added to a PCR reagent. For each target organism to be detected, i.e., for each pathogen to be detected, the PCR reagent comprises at least one specific oligonucleotide primer pair (in short: “primers”), at least one labeled oligonucleotide, which can be used as a probe for detecting the pathogens, and a thermally stable DNA polymerase, for example, a Taq polymerase. The PCR reagent moreover comprises desoxynucleotides, cofactors for the polymerase, and a polymerase buffer solution. In a thermal cycler, the lysate is first heated to approximately 96° C. so that the double-stranded DNA is separated into two individual strands. The primers subsequently hybridize at a lower temperature to the individual strands, and each define a starting point for the following DNA synthesis. The temperature is again increased for the DNA synthesis. The Taq polymerase here fills the free strands with nucleotides. It is, however, also conceivable that the temperature for the DNA synthesis not be increased, and that the DNA synthesis occur at the same temperature as the hybridization of the primers. The described procedure is repeated for a specified number of cycles. If no pathogens are detected after the specified cycles, it can be assumed that the sample is free of the pathogens to be detected.

In order to protect the Taq polymerase and to be able to carry out a PCR investigation, the lysis step is terminated before the detection step. Terminating an enzymatic lysis by increasing temperature has previously been described. If lysis takes place with an enzyme, for example, with a proteinase K, the enzyme can be deactivated by increasing temperature. The present invention avoids the disadvantages associated with the temperature increase, in particular waiting times.

It is conceivable that the method be carried out as a pool testing, i.e., that the multiplex PCR investigation be carried out on a PCR sample comprising several lysates. It is thereby conceivable that the lysates are produced from different samples. It can, for example, be provided for this purpose that the different samples be samples of different foods. It is also conceivable, however, that the different samples be samples of the same foods of different batches.

The present invention provides that the termination of the lysis can, for example, be assisted and/or brought about by a dilution of the lysate, and that the multiplex PCR investigation be carried out on the diluted lysate. It is thereby advantageously possible to terminate the lysis at a constant or even decreasing temperature. It is also conceivable that nucleic acid-free and nuclease-free water be pipetted into the lysate for dilution. “Pipetting” in the context of the present invention is to be understood so that the nucleic acid-free and nuclease-free water is added to the lysate, or that the lysate is added to the nucleic acid-free and nuclease-free water. It can, for example, be provided that the dilution be a dilution of 1:3 to 1:100, for example, 1:10 to 1:75, for example, 1:20 to 1:40. It is also conceivable that a PCR reagent in lyophilized form be dissolved in nucleic acid-free and nuclease-free water, and that the lysate be subsequently pipetted so that the above-mentioned dilution of the lysate is achieved. It has surprisingly been found that dilution leads to termination of the lysis.

It can, for example, alternatively or additionally be provided that the termination of the lysis, and in particular of a lysis facilitated or assisted by a protease, be assisted and/or brought about by adding a protease inhibitor, for example, 4-(2-aminoethyl)-benzene sulfonyl fluoride-hydrochloride (AEBSF), to the lysate. It has surprisingly been found that the addition of protease inhibitors to the lysate stops the lysis. If the lysis step is carried out with the aid of an enzyme, the enzyme is chemically deactivated by the addition of the protease inhibitor to terminate the lysis. It can, for example, be provided that a waiting time of 1 minute to 40 minutes be observed between the lysis step and the detection step. This provides that the lysis is terminated before the lysate comes into contact with the PCR reagent.

In an embodiment of the present invention, it can, for example, be provided that the termination of the lysis be assisted and/or brought about by adding a protease inhibitor, for example, 4-(2-aminoethyl)-benzene sulfonyl fluoride-hydrochloride (AEBSF), to the lysate, and by dilution. It can, for example, be provided that the dilution follow in time after the addition of the protease inhibitor. The multiplex PCR investigation is subsequently carried out on the diluted lysate. The dilution can, for example, be a dilution of 1:3 to 1:100, for example, 1:10 to 1:75, for example, 1:20 to 1:40. The addition of the protease inhibitor and the subsequent dilution bring about a particularly reliable termination of the lysis. If the lysis step is carried out with the aid of an enzymatic lysis with an enzyme, the enzyme is chemically deactivated by the addition of the protease inhibitor to terminate the lysis.

It can, for example, be provided that a waiting time be observed between the addition of the protease inhibitor and the dilution. The protease inhibitor is given sufficient time to chemically terminate the enzymatic lysis as a result thereof.

In an embodiment, the present invention can, for example, provide that the lysate be a crude lysate. A crude lysate is a lysate for which no separation of DNA and residual materials from the lysate has been carried out. Concentration of the DNA does not take place in this case. This advantageously eliminates the need for purification of the lysate. The method is thus significantly more time-saving, less costly, and conserves more medium compared to methods where a purification or concentration with the aid of spin columns or magnetic beads is used. The term “separation” may mean a physical separation. It is, however, also conceivable that “separation” means a binding of the DNA to a surface, for example, a surface of the beads or of a column matrix.

In order to improve the effectiveness of the lysis, an embodiment of the present invention provides that, during the lysis step, a thermal lysis can, for example, first be carried out at a first temperature. The cell walls of the pathogens are thus first broken open by heating. A chemical lysis and/or an enzymatic lysis and/or a mechanical lysis are then subsequently carried out at a second temperature. The second temperature is lower than the first temperature, i.e., the temperature is thus lowered, after the thermal lysis, to start the chemical or enzymatic or mechanical lysis. The cell walls of the pathogens are very reliably broken open by combining a thermal lysis, a chemical lysis, and an enzymatic or a mechanical lysis. The enzymatic lysis can, for example, be carried out with a proteinase K. The mechanical lysis can, for example, be carried out with beads, for example, on a ceramic, glass, or zirconium basis.

An embodiment of the present invention provides that the pre-enrichment medium can, for example, be a non-selective pre-enrichment medium, and that the pre-enrichment can, for example, be a non-selective pre-enrichment. The non-selective pre-enrichment medium can, for example, not have antibiotics. The different pathogens and any pathogenic or non-pathogenic accompanying flora contained in the sample are thus multiplied simultaneously. Accompanying flora in the sense of the present invention comprise micro-organisms which do not belong to the plurality of pathogens.

An embodiment of the present invention alternatively provides that the pre-enrichment medium can, for example, be a selective pre-enrichment medium, and that the pre-enrichment can, for example, be a selective pre-enrichment.

It is in particular provided that the plurality of different pathogens have at least two pathogens from the following group of pathogens: Enterobacteriaceae family, Salmonella genus, Listeria genus, Staphylococcus genus, Shigatoxin-forming E. coli, and Cronobacter genus. It can, for example, be provided that the plurality of different pathogens have the Enterobacteriaceae family, the Salmonella genus, the Listeria genus, the Staphylococcus genus, and Shigatoxin-forming E. coli. It is thus possible to save time and simultaneously perform investigations of pathogens which are legally prescribed for foods.

The present invention also provides a kit. The kit according to the present invention for detecting a plurality of different pathogens is provided for use with the method according to the present invention. The kit according to the present invention has the pre-enrichment medium for the pre-enrichment in the pre-enrichment step. The kit according to the present invention also has a lysis reagent for breaking open the cell walls in the lysis step. The lysis reagent can, for example, have proteinase K. The kit according to the present invention further comprises a PCR reagent for performing the multiplex PCR investigation in the detection step.

In an embodiment, the present invention provides that the kit can, for example, comprise a protease inhibitor, for example, 4-(2-aminoethyl)-benzene sulfonyl fluoride-hydrochloride (AEBSF), for terminating the lysis. It is also conceivable that the kit comprises a lysis buffer.

The kit can, for example, comprise nucleic acid-free and nuclease-free water for diluting the lysate.

The plurality of different pathogens can, for example, comprise Salmonella spp., Listeria spp., Listeria monocytogenes, and Enterobacteriaceae. The plurality of different pathogens can, for example, comprise Cronobacter, coagulase-positive staphylococci, STX/STC, and EAC.

All details, features, and advantages disclosed above in connection with the method according to the present invention likewise relate to the kit according to the present invention.

Further details, features, and advantages of the present invention emerge from the drawing and from the following description of preferred embodiments with reference to the drawing. The drawing merely illustrates an exemplary embodiment of the present invention and in no way restricts the inventive idea.

The FIGURE schematically shows a method according to an embodiment of the present invention. The FIGURE also shows a kit 100 according to an embodiment of the present invention in a highly schematic representation.

In the method shown for detecting a plurality of different pathogens in a sample, the sample is first, in a pre-enrichment step 1, added to a pre-enrichment medium for pre-enrichment. The sample is a 25 g food sample, which is incubated in 225 mL of the pre-enrichment medium at 37° C. for 18 to 30 hours, and in particular 22+/−2 hours. The pre-enrichment medium is here a non-selective pre-enrichment medium for the non-selective pre-enrichment of several pathogens.

After sufficient pre-enrichment, the cell walls of the pathogens in the pre-enriched sample are broken open in a lysis step 2. For this purpose, the pre-enriched sample is first heated to a temperature of 95° C.+/−5° C. and subjected to a thermal lysis. The temperature is subsequently lowered, and reagents for a chemical lysis are added. It is conceivable that an enzyme, for example, a proteinase K, also be added in order to bring about an enzymatic lysis. It is also conceivable for the lysis to be brought about or assisted, additionally or alternatively, by a mechanical lysis. The enzymatic lysis is terminated after an incubation time of 5 to 30 minutes. This takes place while the temperature is maintained or lowered. A protease inhibitor for deactivating the proteinase K is added to the lysate therefor. The lysate is also diluted with nucleic acid-free and nuclease-free water after the addition of the protease inhibitor and a waiting time. The dilution can, for example, be 1:20 to 1:40.

The lysate is a crude lysate in the shown exemplary embodiment.

If the lysis is terminated, i.e., the lysis step 2 has ended, the pathogens are detected in a detection step 3 via a multiplex PCR investigation. The diluted crude lysate is added to a PCR reagent therefor. For each target organism to be detected, i.e., for each pathogen to be detected, the PCR reagent comprises at least one specific oligonucleotide primer pair (in short: “primers”), at least one labeled oligonucleotide, which can be used as a probe for detecting the pathogens, and a thermally stable DNA polymerase, for example, a Taq polymerase. The PCR reagent also comprises desoxynucleotides, cofactors for the polymerase, and a polymerase buffer solution. In a thermal cycler, the lysate is first heated to approximately 96° C. so that the double-stranded DNA is separated into two individual strands. The primers subsequently hybridize at a lower temperature to the individual strands, and each define a starting point for the following DNA synthesis. The temperature is again increased for the DNA synthesis. The Taq polymerase here fills the free strands with nucleotides. It is also conceivable, however, that the temperature for the DNA synthesis is not increased, and that the DNA synthesis occurs at the same temperature as the hybridization of the primers. The described procedure is repeated for a specified number of cycles. If no pathogens are detected after the specified cycles, it can be assumed that the sample is free of the pathogens to be detected.

The kit 100 according to the present invention in this case comprises the non-selective pre-enrichment medium, a lysis reagent, for example, with proteinase K, for example, a lysis buffer, a protease inhibitor for terminating the enzymatic lysis, nucleic acid-free and nuclease-free water for diluting the crude lysate, and the PCR reagent.

The present invention is not limited to embodiments described herein; reference should be had to the appended claims.

### LIST OF REFERENCE NUMERALS

1 Pre-enrichment step
2 Lysis step
3 Detection step
100 Kit

What is claimed is:

1. A method for detecting a plurality of different pathogens in a sample, the method comprising:
a pre-enrichment step comprising adding the sample to a pre-enrichment medium so as to obtain a pre-enriched sample;
a lysis step comprising breaking open cell walls of the plurality of different pathogens in the pre-enriched sample so as to provide a lysate; and
a detection step comprising detecting the plurality of different pathogens via a multiplex PCR investigation,
wherein,
the pre-enrichment step, the lysis step, and the detection step are performed in an order set forth, and
the lysis step is terminated before the detection step without increasing a temperature.

2. The method as recited in claim 1, wherein,
a termination of the lysis step is at least one of assisted by and brought about by a dilution of the lysate so as to obtain a diluted lysate,
the multiplex PCR investigation in the detection step is performed on the diluted lysate, and
the dilution is from 1:3 to 1:100.

3. The method as recited in claim 1, wherein the termination of the lysis step is at least one of assisted by and brought about by adding a protease inhibitor to the lysate.

4. The method as recited in claim 3, wherein the protease inhibitor is 4-(2-aminoethyl)-benzene sulfonyl fluoride-hydrochloride.

5. The method as recited in claim 1, wherein,
a termination of the lysis step is at least one of assisted by and brought about by first adding a protease inhibitor to the lysate followed by a dilution of the lysate so as to obtain a diluted lysate,
the multiplex PCR investigation in the detection step is performed on the diluted lysate, and
the dilution is from 1:3 to 1:100.

6. The method as recited in claim 5, wherein,
the protease inhibitor is 4-(2-aminoethyl)-benzene sulfonyl fluoride-hydrochloride, and
the dilution is from 1:10 to 1:75.

7. The method as recited in claim 5, wherein a waiting time exists between the adding of the protease inhibitor and the dilution.

8. The method as recited in claim 1, wherein the lysate is a crude lysate.

9. The method as recited in claim 1, wherein the lysis step (2) comprising breaking open cell walls of the plurality of different pathogens in the pre-enriched sample so as to provide the lysate includes,
first preforming a thermal lysis at a first temperature, and
subsequently performing at least one of a chemical lysis, an enzymatic lysis, and a mechanical lysis at a second temperature,
wherein,
the second temperature is lower than the first temperature.

10. The method as recited in claim 1, wherein,
the pre-enrichment medium is a non-selective pre-enrichment medium, and
the pre-enrichment step provides a non-selective pre-enrichment of the sample.

11. The method as recited in claim 1, wherein the plurality of different pathogens include at least two pathogens from the following group of pathogens,
the Enterobacteriaceae family,
the Salmonella genus,
the Listeria genus,
the Staphylococcus genus,
Shigatoxin-forming E. coli, and
the Cronobacter genus.

12. The method as recited in claim 1, wherein the plurality of different pathogens include at least two pathogens from the following group of pathogens,
the Enterobacteriaceae family,
the Salmonella genus,
the Listeria genus,
the Staphylococcus genus, and
and Shigatoxin-forming E. coli.

13. A kit for detecting a plurality of different pathogens using the method as recited in claim 2, the kit comprising:
a pre-enrichment medium for providing the pre-enriched sample in the pre-enrichment step;
a lysis reagent for breaking open the cell walls of the plurality of different pathogens in the lysis step; and
a PCR reagent for performing the multiplex PCR investigation in the detection step.

14. The kit as recited in claim 13, further comprising:
a protease inhibitor for the termination of the lysis step.

15. The kit as recited in claim 14, wherein the protease inhibitor is 4-(2-aminoethyl)-benzene sulfonyl fluoride-hydrochloride.

16. The kit as recited in claim 13, further comprising a nucleic acid-free and nuclease-free water for the dilution of the lysate.
